(19) United States
(12) Patent Application Publication
(10) Pub. No.: US 2011/0189667 A1
(43) Pub. Date: Aug. 4, 2011

(54) METHOD OF SCREENING FOR NOVEL EXON 1 MUTATIONS IN MECP2 ASSOCIATED WITH CLASSICAL RETT SYNDROME

(75) Inventor: Carol Saunders, Kansas City, MO (US)

(73) Assignee: The Children's Mercy Hospital, Kansas City, MO (US)

(21) Appl. No.: 12/863,078

(22) PCT Filed: Jan. 16, 2009
(86) PCT No.: PCT/US09/31271

§ 371 Date: Feb. 16, 2011

Related U.S. Application Data
(60) Provisional application No. 61/021,413, filed on Jan. 16, 2008.

(51) Int. Cl. C12Q 1/68

(52) U.S. Cl. 435/6.11

(57) ABSTRACT

Recently, a new MECP2 isoform, which has an alternative N-terminus, transcribed from exon 1, was described. Since the incorporation of exon 1 into standard sequencing protocol for Rett syndrome, few patients with exon 1 mutations have been described and several groups have concluded that exon 1 mutations are a rare cause of Rett syndrome. The present invention provides an improved method of diagnosing Rett Syndrome by identifying two different mutations in exon 1 of the MECP2 gene, the first of which results in a switch from alanine to valine at the beginning of a polyalanine stretch, and the second of which results in a disruption of the ATG initiation codon of exon 1. Patients having either such mutation fit the clinical criteria for classic Rett syndrome, and further support previous reports that exon 1 mutations may be associated with a severe phenotype.

### BACKGROUND

Rett syndrome (RTT) is a progressive neurological developmental disorder affecting 1:8,000 girls, making it the most common genetic cause of profound mental retardation in females (Laurvick et al., 2006). RTT is caused by de novo mutations in the X-linked MECP2 gene, which was cloned in 1996 and first described as a three-exon gene initiating from an ATG in exon 2 (Amir et al., 1999). Two recent studies have described a new MECP2 splice variant, MECP2_e1, which is transcribed from an initiation codon in exon 1 and omits exon 2 through alternative splicing (Kriaucionis and Bird, 2004, Mnatzakanian et al., 2004). Since exon 1 was previously thought to be noncoding, it was excluded from most sequencing protocols until recently. By sequencing just exons 2-4, the detection rate in patients with classic RTT is ˜85%, with another ˜5-10% harboring large deletions detectable only by dosage sensitive methods such as MLPA (Shahbazian M D and Zoghbi H Y, 2001, Schollen et al, 2003). Re-screening of exon 1 in patients previously negative for mutations in exons 2-4 of MECP2 has yielded low detection rates (Amir et al., 2005, Bartholdi et al., 2006, Quenard et al., 2006), with only 10 patients testing positive for mutations in exon 1 by sequencing methods. Additionally, 2 patients were reported with whole exon deletions exclusive to exon 1, which would have previously been dismissed as non-pathogenic since they do not involve the coding region of the original isoform, MECP2_e2 (Mnatzakanian et al., 2004, Quenard et al., 2006). Here the exon 1 mutation rate is assessed in a small, unselected set of samples for RTT testing and the associated clinical phenotypes in those who tested positive were reported.

### SUMMARY OF THE INVENTION

The present invention describes a novel mutation causing a C>T transition (c.5C>T) resulting in a point mutation, Alanine to Valine (A2V), discovered in a unique region of exon 1 of MECP2. This mutation is shown in SEQ ID NO. 2, wherein it can be seen that this sequence varies from SEQ ID NO. 1 at a single position. This mutation is shown in the first triplet after the ATG start codon wherein the gcc found in the wild-type sequence has been mutated to gtc, thereby resulting in the A2V mutation. This is the first point mutation discovered in exon 1. Such a mutation can exist anywhere within the polyalanine stretch. The present invention also describes another mutation of a single base pair in exon 1, c.1A>T (p.Met1?), which results in an alteration of the “A” of the initiation codon (ATG), most likely disrupting translation MECP2_e1. This mutation is shown in SEQ ID NO. 3, which differs from SEQ ID NO. 1 in the ATG start (or initiation) codon which has been mutated to ttg. These discoveries were unexpected in light of the fact that exon 1 had not previously been included as a region of interest when conducting diagnostic testing for RTT. These mutations and the regions in which they are located have several applications, including diagnostic testing, diagnostic kits, identification of a region of interest for new drug applications, and gene therapy.

All mutations localized to exon 1 reported to date have been either small insertions or deletions or large deletions removing the entire exon. These two single base pair changes are the first point mutations to be reported in exon 1 of the MECP2 gene. The c.1A>T mutation alters the initiation codon, which would mostly likely result in absent translation of MECP2E1. MECP2E2 would be presumably unaffected but is clearly unable to compensate, as evidenced by the classic RTT symptoms exhibited by an individual with this mutation. As noted above, the novel C>T transition (c.5C>T) resulted in a point mutation, A2V. This alanine is a perfectly conserved residue that marks beginning of a polyalanine stretch that is present in all species. (Harvey et al., 2007). The role of this repeat is unknown, but it could play a role in the regulation of gene transcription, given the multiple binding sites for the SP1 transcription factor. The parents of the individual having this mutation both tested negative for this mutation, indicating that this is a de novo, most likely pathogenic, mutation.

Previous studies concluded that sequencing exon 1 contributed little to the mutation detection rate in RTT, even in pre-selected populations such as classical patients who had already tested negative for mutations in exons 2-4 of the gene (Amir et al., 2005, Evans et al., 2005, Quenard et al. 2006). The experience of this clinical laboratory is quite different: In a span of two years, a total of 35 female patients were tested, a minority of whom met the clinical criteria for classical RTT (7 individuals) or variant RTT (2 individuals). Other clinical presentations such as autism or developmental delay were much more frequent in the testing population, which would be less likely to be associated with a MECP2 mutation. Seven other studies examining the exon 1 mutation frequency have been published to date (see Table 2). All of the previous studies were restricted to patients meeting criteria for classic or variant RTT and except for one study (Quenard et al., 2006), all were looking at patients who had previously tested negative for mutations in exons 2-4.

Although genotype-phenotype correlations are difficult to make in RTT because of differences in X-chromosome inactivation, several authors have observed that patients with exon 1 mutations result in a severe RTT phenotype (Amir et al., 2005, Bartholdi et al., 2006, Chunshu et al., 2006). This could be because exon 1 mutations cause premature truncation of the more relevant, brain-dominant isoform (Kriaucionis and Bird, 2004, Mnatzakanian et al., 2004). Out of 13 patients harboring mutations within exon 1, all but two had classic/severe RTT. The two patients with atypically mild RTT had the same c.47_{—}57del11nt mutation, which has also been reported in classic RTT patients (Table 1), differences which could be attributed to skewed XCI. All three patients in the study had classic RTT, with one dying an early death from pneumonia at the age of 16. It is worth noting that 4 of the 13 patients listed in Table 1 died by the age of 25 (median age 17.5). RTT patients do have a decreased survival compared to the general population, but survival to 20 years was 94% in a preliminary study of patients from Texas (del Junco et al., 1993) and 85.3% in a large Australian cohort of 276 RTT patients (Laurvick et al., 2006). Interestingly, when the data from the Australian group was stratified according to whether the patient tested positive for a MECP2 mutation, the survival for mutation-confirmed RTT patients was 77.8% (95% CI 66.8-85.6%) by 25 years versus 56.5% (95% CI 17.3-83%) in those without identifiable mutations. Since the mutation analysis excluded exon 1, any patients with mutations in exon 1 would have been in the group with lower survival. More studies are needed to determine whether mortality differs according to genetic mutation and whether exon 1 is a risk factor for early death.

The present invention discloses a method of diagnosing Rett Syndrome. The method includes obtaining a sample containing DNA from a patient. The preferred sample is blood but can include most any tissue type. After the DNA is obtained, analysis of the MECP2 region, including exon 1, is performed. Preferably, the analysis includes amplifying the exon 1 region and then verifying on a 2% agarose gel. In a more preferred embodiment, the fragments are purified preferably using ExoSAPit (USB) or the like, and those products are bidirectionally sequenced using, for example, an automated fluorescent dye-terminator sequencing using Big Dye v3.0 (Applied Biosystems) and run on an ABI310 (Applied Biosystems, Foster City, Calif.) or other capillary electrophoresis instrument. The patient's DNA is then screened for a point mutation in exon 1. If such a mutation in exon 1 is identified, the patient is diagnosed as having Rett syndrome. More preferably, the analysis will screen for a mutation that results in a switch from an alanine to valine, preferably in a polyalanine stretch, and even more preferably at the beginning of a polyalanine stretch. Most preferably, such a mutation will result in the alanine to valine switch present when comparing SEQ ID NO. 1 with SEQ ID NO. 2, wherein SEQ ID NO. 1 represents a non-mutated sequence and SEQ ID NO. 2 includes the alanine to valine mutation at the beginning of a polyalanine stretch. As will be understood by those of skill in the art, any method that is capable of accurately sequencing exon 1 of MECP2 can be used for the analysis. The resulting sequence is then compared with a sequence known to not include any such mutation and differences between the two sequences are noted.

In another embodiment of the present invention, a method for screening an individual for a novel point mutation in exon 1 is disclosed. A sample is collected from a patient and DNA sequenced from the exon 1 region of MECP2, preferably according to the method described above. The sequenced DNA is then screened for a point mutation in exon 1.

In another embodiment of the present invention, a method for screening an individual for a novel missense mutation is disclosed. A DNA sample is collected from the patient and then sequenced, according to the method above. The sequenced DNA is then screened for a missense mutation in exon 1.

In another embodiment of the present invention, a method for screening an individual for a C>T transition (c.5C>T) resulting in a point mutation, A2V, is disclosed. A sample is obtained from a patient and the DNA sequenced, according to the method above. The sequenced DNA is screened for a C>T transition (c.5C>T) resulting in a point mutation, A2V.

In yet another embodiment of the present invention, a method of screening for Rett syndrome is disclosed. A sample from the patient is taken and the DNA sequenced, according to the method above. The sequenced DNA is then screened for a point mutation wherein there is a C>T transition (c.5C>T), namely, the A2V substitution.

In another embodiment, a method of diagnosing Rett syndrome is disclosed wherein a patient exhibiting at least one symptom associated with classical Rett Syndrome is selected and a sample taken. The sample containing DNA is then sequenced and analyzed according to the methods disclosed above.

### DETAILED DESCRIPTION

The following examples are provided for illustrative purposes only. Nothing contained herein shall be construed as a limitation of the scope of the present invention. Additionally, the teachings and content of all references cited herein are hereby incorporated by reference herein.

### Example 1

### Materials and Methods

### Patients

35 samples from females were referred for RTT testing in a two year period spanning September of 2004 through September of 2006. These patients had various clinical presentations, including autism, mental retardation, developmental delay, and “Angelman-like” symptoms. Furthermore, only 9 patients fit the criteria for classical (7) or variant (2) RTT. 4 patients had previously tested negative for mutations in exons 2-4 and were therefore tested only for exon 1. Permission to review patient charts was obtained through the Children's Mercy Hospitals and Clinics' Institutional Review Board.

Patient 1 was a 20-year-old at the time of testing who had a long standing clinical diagnosis of RTT but had never undergone confirmatory DNA testing. She met the criteria for classical RTT, with the exception of acquired microcephaly (head circumference is at 15%). Following normal perinatal development, she sat at 6 months, walked at 14 months, and used simple words at 18 months, around which time she began to regress. She lost all speech in addition to purposeful hand movements, which were replaced by a sifting activity. She now walks with a shuffling gait, exhibits some aggressive behavior, is nonverbal, and has medically intractable epilepsy.

Patient 2 was 7 at the time of testing. She met the criteria for classical RTT, with the exception of acquired microcephaly (head CT at 50%). She had some period of normal development, such as smiling, rolling over, and sitting at appropriate times, but around 10 months she exhibited global developmental delay. There was no clear regression in her skills. Around the age of 2, she developed a stereotypic midline hand movement involving her left hand in her mouth and her right hand twirling her hair or rubbing her hair between her fingers. She commando crawls for mobility and will take steps with assistance. She is very hirsute and has precocious puberty with pubic hair development beginning at age 5. She has episodic seizures that do not require daily medication. She had previously tested negative for MECP2 mutations in exons 2-4, MECP2 duplications, MECP2 deletions, and research testing involving sequencing of the MECP2 promoter region. The family came to the clinic in pursuit of CDKL5 sequencing, but upon closer examination of the patient's medical record, it was discovered that exon 1 had not been tested.

Patient 3 was a 16-year-old female with a clinical diagnosis of Rett syndrome since 20 months of age. She had microcephaly, developmental regression, severe cognitive insufficiency, midline hand movements, general tonic-clonic seizure disorder, loss of gait, diffuse hypertonicity, scoliosis treated with surgery, GE reflux requiring gastrostomy tube, and multiple hospitalizations for bacterial pneumonia. On her last admission for pneumonia, she succumbed to respiratory insufficiency. A brain autopsy showed microcencephaly, subpial gliosis, minimal loss of Purkinje cells with gliosis, and isolated eosinophilic neurons in the dentate nucleus and brain stem. Previous testing for MECP2 exons 2-4 was negative.

### Sequence Analysis

DNA from blood, or in the case of patient 3, cultured fibroblast cells, was extracted by a manual salting out procedure (Lahiri et al., 1991). For Patient 1, the entire MECP2 coding region (exons 1-4) was analyzed; for Patients 2 and 3, only exon 1 was analyzed since the remaining coding region had been previously tested by an outside laboratory. Exon 1 of the MECP-2 gene was PCR-amplified as described (Mnatzakanian et al., 2004) and verified on a 2% agarose gel. Fragments were purified using ExoSAPit (USB). Purified products were bidirectionally sequenced by automated fluorescent dye-terminator sequencing using Big Dye v3.0 (Applied Biosystems) and run on an ABI310 (Applied Biosystems, Foster City, Calif.). For Patient 2, single stranded sequence was obtained after cloning the heterozygous PCR product into a TA cloning vector (Invitrogen). The sequence data was compared to the MECP2 reference sequence AF030876 using Sequencher software.

### Results

In 35 samples tested for RTT, three unrelated patients with exon 1 mutations, one of which was novel, were reported.

In Patient 1, we detected a mutation, c.1A>T that disrupts the initiation codon, changing it to a leucine. The prediction is that MECP2E1 translation would be greatly or totally hindered due to absence of a start codon. MECP2E2 would be presumably unaffected and is unable to compensate. The patient's mother tested negative for this mutation.

Patient 2 has a previously reported mutation, c.62+1delTG, affecting the splice donor. (Amir et al., 2005). Analysis of parental DNA revealed that it arose as a de novo mutation, not present in either parent. This mutation is predicted to disrupt splicing of the MECP2e1 isoform, and may also affect the expression of the exon 2-containing product, MECP2e2 (Amir et al., 2005, Saxena et al., 2006). This patient has a random pattern of X-chromosome inactivation in peripheral blood leukocytes.

Patient 3 had a novel C>T transition (c.5C>T) resulting in a point mutation, A2V. Though an alanine to valine substitution is conservative in retaining a nonpolar side chain, this is a residue that is perfectly conserved throughout evolution and marks the beginning of a polyalanine stretch which is present in all species. (Harvey et al., 2007). Though the role of this repeat is unknown, it contains multiple binding sites for the SP1 transcription factor, the alterations of which would affect the rate of gene transcription. This patient's parents both tested negative for this mutation, indicating this is a de novo, most likely pathogenic mutation.

Three mutations within exon 1 of the MECP2 gene were detected in 35 clinical samples referred for MECP2 sequencing. Two of these mutations were novel and one previously reported; all three were associated with classical RTT. Two of these patients had previously tested negative by molecular testing, which at the time included sequencing exons 2-4 of the MECP2 gene. Following the reports of the second MECP2 isoform and the clinical utility of sequencing exon 1, these patients were tested for exon 1 mutations. The three mutations reported here bring the total number of distinct exon 1 mutations detected by sequencing to 9. Two of these mutations, c.47_{—}57del11nt and c.62+1delGT, have been found in more than one patient (see Table 1), including Patient 2 of this report. This brings the number of patients harboring a mutation within exon 1 of MECP2 to 13.

TABLE 1
Summary of exon 1sequence mutations reported in MECP2 to date:

| | Patient | Age at Death | | | |
|---|---|---|---|---|---|
| Mutation | Age | (Cause) | XCI | Phenotype | Reference |
| c.1A > T | 20 | n/a | Not | classic RTT | This study |
| | | | done | | |
| c.5C > T | | 16 | Not | classic RTT | This study |
| | | (pneumonia) | done | | |
| c.23_27dup5nt | | 25 (not | — | classic RTT | Ravn et al., 2005 |
| | | given) | | | |
| c.30delCinsGA | | 19 | 70:30 | classic RTT | Bartholdi et al., |
| | | (pneumonia) | | | 2006 |
| c.47_57del11nt | 27 | n/a | — | classic RTT | Mnatzakanian et |
| | | | | | al., 2004 |
| c.47_57del11nt | 37 | n/a | — | classic RTT | Ravn et al., 2005 |
| c.47_57del11nt | ? | n/a | 44:56 | atypical (mild) RTT | Amir et al., 2005 |
| c.47_57del11nt | 13 | n/a | 73:27 | atypical (mild) RTT | Saxena et al., |
| | | | | | 2006 |
| c.48_55dup | 5 | n/a | Random | classic RTT | Quenard et al. |
| | | | | | 2006 |
| c.59_60delGA | 5 | n/a | 48:52 | classic RTT | Chunshu et al., |
| | | | | | 2006 |
| c.62 + 1delGT | 8 | n/a | 68:32 | classic RTT | Amir et al., 2005 |
| c.62 + 1delGT | 7 | n/a | 78:22 | classic RTT | This study |
| c.62 + | | 6½ (not | Random | atypical (severe) | Quenard et al. |
| 2_62 + 3del | | given) | | RTT | 2006 |

The detection rates for mutations within exon 1 range from 0% to 25% (See Table 2) in these studies, with several groups concluding that exon 1 mutations are a rare cause of RTT (Amir et al., 2005, Evans et al., 2005, Quenard et al. 2006). In our group of 35 unselected patients, 3 had exon 1 mutations (8.6%). For the sake of comparison, if we restrict our numbers to only those patients who fit the classic or atypical RTT criteria, our exon 1 mutation frequency is 33%. The average detection rate from the reports listed in Table 2 is 8.1% (median 5%). Taken together, these data indicate that exon 1 mutations detectable by sequencing are slightly more common that previously reported (Amir et al., 2005, Evans et al., 2005, Quenard et al. 2006).

TABLE 2
Literature Reports of Exon 1 Mutation Detection

| | | | | Large Gene |
|---|---|---|---|---|
| | | | | Rearrange- |
| | Frequency of | | Previously | ments |
| | Mutations | | Negative for | Including |
| Study | within Exon 1 | Phenotype | Exons 2-4 | Exon 1 |
| Mnatzakanian et | 1/19; 5.2% | Typical RTT | Yes | 1 patient, |
| al., 2004 | | | | exon 1 |
| Amir et al., | 2/63; 3.2% | 38 classic RTT, 25 | Yes | Not tested |
| 2005 | | atypical RTT | | |
| Quenard et al., | 2/212; .9% | 211 typical RTT, 1 | No | 4 patients, |
| 2006 | | atypical (severe) RTT | | large |
| | | | | deletions* |
| Ravn et al., | 2/10; 20% | Typical RTT | Yes | None |
| 2005 | | | | |
| Bartholdi, et al., | 1/20; 5% | 12 classic RTT, 8 | Yes | 1 patient, |
| 2006 | | variant RTT, | | exons 1-2 |
| Saxena et al., | 1/20; 5% | Classic and atypical | Yes | Not tested |
| 2006 | | RTT | | |
| Evans et al., | 0/97; 0% | 37 classic RTT and 60 | Yes | None (Not all |
| 2005 | | atypical | | were tested) |
| Chunshu et al., | 1/4; 25% | Classic RTT | Not specified | n/a |
| 2006 | | | | |
| This study | 3/35; 8.6% | 7 classical RTT, 2 | 5 Patients | Not tested |
| | | variant RTT; (The rest | | |
| | | have autism, MR, | | |
| | | microcephaly, etc.) | | |
| Total | 13/480; 2.7% | | | 6 Deletions; |

*One promoter and exon 1, one exons 1-2, one promoter and exons 1-2, and one complete gene deletion

### REFERENCES

All references cited herein are incorporated by reference in their entireties.
Amir, R E, Fang P, Yu Z, Glaze D G, Percy A K, Zoghbi H Y, Roa B B, Van den Veyver I B, 2005. Mutations in exon 1 of MECP2 are a rare cause of Rett syndrome. J Med Genet 42:e15.
Amir R E, Van den Veyver I B, Wan M, Tran c Q, Francke, U, Zoghbi, H Y, 1999. Rett syndrome is caused by mutations in X-linked MECP2, encoding methyl-CpG-binding protein 2. Nat Genet 23: 185-188.
Bartholdi D, Klein A, Weissert M, Koenig N, Baumer A, Boltshauser E, Schinzel A, Berger W, Matyas G, 2006. Clinical profiles of four patients with Rett syndrome carrying a novel exon 1 mutation or genomic rearrangement in the MECP2 gene. Clin Genet 69: 319-326.
Chunshu Y, Endoh K, Soutome M, Kawamura R, Kubota T, 2006. A patient with classic Rett syndrome with a novel mutation in MECP2 exon 1. Clin Genet 70: 530-531.
Evans J C, Archer H L, Whatley S D, Kerr A, Clarke A, Butler R, 2005. Variation in exon 1 coding region and promoter of MECP2 in Rett syndrome and controls. Eur J Hum Genet 13: 124-126.
Harvey C G, Menon S C, Stachowiak B, Noor A, Proctor A, Mensah A K, Mnatzakanian G C, Alfred S E, Guo R, Scherer S W, Kennedy J L, Roberts W, Srivistava A K, Minassian B A, Vincent J B, 2007. Sequence Variants Within Exon 1 of MECP2 Occur in Females With Mental Retardation. Am J Med Genet 144B:355-360.
Kriaucionis S and Bird A, 2004. The major form of MECP2 has a novel N-terminus generated by alternative splicing. Nucleic Acids Res 32(5) 1818-1823.
Laurvick C L, de Klerk N, Bower C, Christodoulou J, Ravine D, Ellaway C, Williamson S, Leonard H (2006) Rett syndrome in Australia: a review of the epidemiology. J Pediatr 148:347-52.
Lahiri D K, Nurnberger J I, 1991. A rapid non-enzymatic method for the preparation of HMW DNA from blood for RFLP studies. Nucleic Acids Res. October 11; 19(19):5444.
Mnatzakanian G, Lohi H, Munteanu I, Alfred S, Yamada T, MacLeod P J M, Jones J R, Scherer S W, Schanen C N, Friez, M J, Vincent J B, Minassian B A, 2004. A previously unidentified MECP2 open reading frame defines a new protein isoform relevant to Rett syndrome. Nat Genet 36(4): 339-341.
Quenard A, Yimaz S, Fontaine H, Bienvenu T, Moncla A, des Portes V, Rivier F, Mathieu M, Raux G, Jonveaux P, Philippe C, 2006. Deleterious mutation in exon 1 of MECP2 in Rett syndrome. Eur J Med Genet 49: 313-322.
Ravn K, Nielsen, J B, Schwartz, M, 2005. Mutations found within exon 1 of MECP2 in Danish patients with Rett syndrome. Clin Genet 67:532-533.
Saxena A, de Lagarde D, Leonard H, Williamson S L., Vasudevan, V, Christodoulou J, Thompson E, Macleod P, Ravine D, 2005. Lost in translation: translational interference from a recurrent mutation in exon 1 of MECP2. J Med Genet
Schollen E, Smeets E, Deflem E, Fryns J P, Matthijs G, 2003. Gross rearrangements in the MECP2 gene in three patients with Rett syndrome: implications for routine diagnosis of Rett syndrome. Hum Mutat 22:116-20.
Shahbazian M D and Zoghbi H Y, 2001. Molecular genetics of Rett syndrome and clinical spectrum of MECP2 mutations. Curr Opin Neurol 14:171-6.

We claim:

1. A method of diagnosing Rett Syndrome comprising the step of analyzing exon 1 of the MECP2 gene for a mutation resulting in a switch from an alanine to valine.

2. The method of claim 1, said alanine to valine switch occurring in a stretch of polyalanine residues.

3. The method of claim 1, said alanine to valine switch occurring at the beginning of a polyalanine stretch.

4. The method of claim 1, said mutation being present in SEQ ID NO. 2, but not in SEQ ID NO. 1.

5. A method of diagnosing Rett Syndrome comprising the step of analyzing exon 1 of the MECP2 gene for a mutation resulting in a disruption of the ATG initiation codon.

6. The method of claim 5, said mutation resulting in the ATG initiation codon of exon 1 being mutated to TTG.

7. The method of claim 5, said mutation being present in SEQ ID NO. 3.


SEQUENCE LISTING (3 sequences)

SEQ ID NO: 1 - DNA, 1504 bp, Homo sapiens
ccggaaaatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga

SEQ ID NO: 2 - DNA, 1504 bp, Homo sapiens
ccggaaaatggtcgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga

SEQ ID NO: 3 - DNA, 1504 bp, Homo sapiens
ccggaaattggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga